(19) United States
(12) Patent Application Publication
(10) Pub. No.: US 2006/0219800 A1
(43) Pub. Date: Oct. 5, 2006

(54) Universal bottle for plug-in scented oil

(76) Inventor: Junwu Liu, Changzhou City (CN)

Correspondence Address: LAU & ASSOCIATES, 7701 ROCKLEDGE COURT, SPRINGFIELD, VA, 22152, US

(21) Appl. No.: 11/138,274

(22) Filed: May 27, 2005

(30) Foreign Application Priority Data
Mar. 25, 2005 (CN) 200520070221.2

(51) Int. Cl. A61L 9/04; A24F 25/00

(52) U.S. Cl. 239/44

(57) ABSTRACT

The present utility model relates to a removable liquid bottle, consisting of a bottle for holding liquid, a wick inside the bottle for drawing liquid, a neck in the upper part of the bottle forming an opening at one end; a screw thread tapped on the neck; a stopper, set at the opening, cooperatively working with the wick for sealing purpose; an axial fixation collar, of which the outer diameter is larger than the diameter of the screw thread, located at a lower part of the neck than the screw thread; an projection with a bevel and said projection with a bevel is a wedge with a narrow top and a wide bottom in the direction of the height of the bottle; a fixation projection on the rear external surface of the bottle. The present utility model sets several fixation surfaces which can be used to connect the liquid bottle to a vaporization device of any structure and enhance the removability of the bottle and convenience for the customer.

### TECHNICAL FIELD

The present utility model relates to a vaporization device for vaporizing liquid perfume or antidote and particularly to a removable liquid bottle which can be installed to a vaporization device.

### BACKGROUND

Following the continuous development of technology and enhancement of human life, people are paying more and more attention to the air quality in living and working places. As a product for adjusting and cleaning air, liquid perfume is very popular. The perfume spreads into the air through an atomizer of the bottle and adjusts or cleans the air with the air flow. But using an atomizer suffers from several drawbacks, such as inefficient use of the perfume, short-lasting period, and lack of continuous effect to clean or disinfect the air. The vaporization device for perfume can overcome the above-identified drawbacks. Such vaporization device, including an electric heater and a liquid bottle, can vaporize the perfume in the air by heating perfume drops with the wick. The vaporized perfume can spread to the surrounding environment with the air flow and last a relatively longer period. The liquid bottles are tailored for its corresponding vaporization device and after depleted, the empty bottles can be easily displaced with a bottle full of perfume. However, as a vaporization device of a brand can only interact with a bottle of certain structure, the customers cannot install a perfume of a brand they own to a vaporization device of another brand due to the different structures of the bottles. As a result, liquid bottles suffer from a poor removability to vaporization devices of various and cannot meet the customers' needs.

### OBJECT OF THE INVENTION

The present utility model is to provide a removable liquid bottle with reasonable structure and good removability, which could be installed to vaporization devices of different structures.

The present utility model utilizes the following technical plan: A removable liquid bottle, including a bottle for holding liquid, a wick inside the bottle, a neck in the upper part of the bottle forming an opening at one end; a screw thread tapped on the neck; a stopper, set at the opening, cooperatively working with the wick for sealing purpose; an axial fixation collar, of which the outer diameter is larger than the diameter of the screw thread, located at a lower part of the neck than the screw thread; an projection with a bevel and said projection with a bevel is a wedge with a narrow top and a wide bottom; a fixation projection on the rear external surface of the bottle.

The present utility model enjoys a simple but reasonable structure by setting a screw thread for connection and an axial fixation collar on the neck of the bottle, a projection with a bevel on the front external surface of the bottle and a fixation projection on the rear external surface. Thus, the liquid bottle can be easily connected to a vaporization device of any structure through the axial fixation collar or the screw thread on the neck, the projection with a bevel on the front external surface, the fixation projection on the rear external surface, or the fixation projection in conjunction with the projection with a bevel. Both removability and convenience for the customers are increased.

### DESCRIPTION FOR THE ATTACHED DRAWINGS

The following is a further description of the present utility model with the drawings and actual application.

FIG. 1 is a front view of the removable liquid bottle of the present utility model.

FIG. 2 is a plane view of FIG. 1.

FIG. 3 is a cutaway view of A-A of FIG. 1.

FIG. 4 is a side view of FIG. 1.

FIG. 5 is a cutaway view of B-B of FIG. 4.

Wherein: 1—bottle cap; 2—liquid bottle; 2-1—bottle neck; 2-2—axial fixation collar; 2-3—screw thread; 2-4—projection with a bevel; 2-5—fixation projection; 3—stopper; 3-1—internal housing; 3-2—connecting body; 3-3 external housing; 3-4—outlet; 4—wick.

### DESCRIPTION

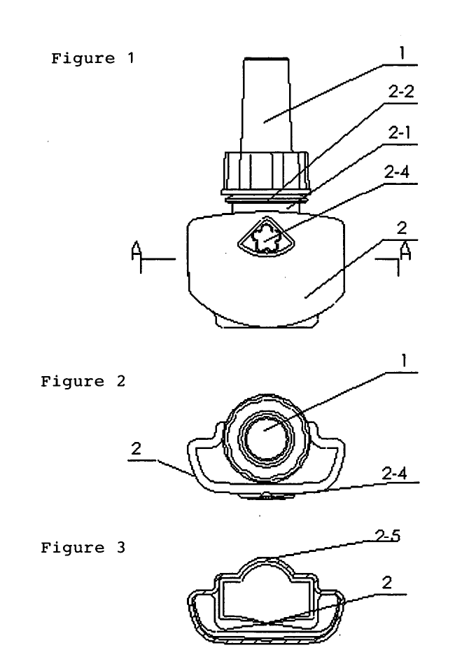

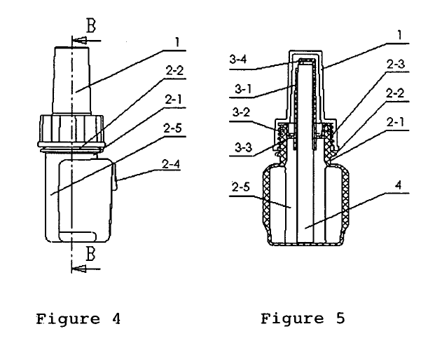

Referring now to FIG. 1 through FIG. 4, there is shown a removable liquid bottle including a bottle (2) for holding liquid and a wick (4) inside bottle (2); a neck (2-1) in the upper part of the bottle (2) forming an opening at one end; a screw thread (2-3) on the neck (2-1) can fit the cap (1) by screwing, or directly fit a vaporization device by screwing in order to connect the bottle to the vaporization device. Stopper (3), which is placed at the opening of the bottle cooperatively with the wick (4) for sealing purpose, consists as shown in FIG. 5 an internal housing (3-1) with the wick (4), an external housing (3-2) grasping the opening of the bottle and a connecting body (3-3) connected between the internal housing (3-1) and the external housing (3-2); the internal housing (3-1) is a cone with a narrow top and a wide bottom with an outlet (3-4) at the top of the internal housing (3-1); The vaporization device heats the internal housing (3-1) of stopper (3) and vaporize the liquid inside wick (4). As shown in FIG. 5, the present utility model fits the front section of a vaporization device by an axial fixation collar (2-2), of which the outer diameter is larger than the diameter of the screw thread, located at a lower part of the neck (2-1) than the screw thread (2-3), in order to connect the liquid bottle to the vaporization device. As shown in FIGS. 1, 2 and 4, there is the projection with a bevel (2-4) on the front external surface of bottle (2), which is a wedge with a narrow top and a wide bottom in the direction of the height of the bottle; preferably, the projection with a bevel (2-4) is in the upper part of bottle (2) so as to fasten and lock the bottle. As shown in FIG. 3, there is a fixation projection (2-5) on the rear external surface of bottle (2); the fixation projection (2-5) is above the convexity of the rear external surface and its height is smaller than or equal to that of bottle (2); thus the fixation projection (2-5) of the bottle (2) and its opposite front section are not centrosymmetric. When the bottle is installed to a vaporization device, it is connected to the device by the projection with a bevel (2-4) on the front external surface of the bottle and the fixation projection (2-5) on the rear external surface; or only by the fixation projection (2-5); or directly by the projection with a bevel (2-4) into the alignment hole on the vaporization device. Preferably, the bottom of the projection with a bevel (2-4) is an arcuate surface as shown in FIG. 1, or a flat surface, by which to position the bottle in the alignment hole of the vaporization device. The bottom can also be a curve which has two minimum points to position the bottle in the alignment hole. Based on the alignment holes of vaporization devices, the shape of projection with a bevel (2-4) and the structure of the bottom can vary a lot so as to be installed to vaporization devices of different structures.

1. A removable liquid bottle, comprising a bottle (2) for holding liquid, a wick (4) inside the bottle (2), a neck (2-1) in an upper part of the bottle (2) having an opening at one end, a screw thread (2-3) tapped on the neck (2-1), and a stopper (3), set at the opening, cooperatively working with the wick (4) for a sealing purpose, is characterized by the fact that: an axial fixation collar (2-2), of which the outer diameter is larger than the diameter of the screw thread, located at a lower part of the neck (2-1) than the screw thread (2-3); a projection with a bevel (2-4) on the front external surface of the bottle (2) and said projection with a bevel (2-4) is a wedge with a narrow top and a wide bottom; a fixation projection (2-5) on the rear external surface of the bottle (2).

2. A removable liquid bottle as described in claim 1, is characterized by the fact that: said fixation projection (2-5) of the bottle (2) is a convex surface above the rear external surface.

3. A removable liquid bottle as described in claim 2, is characterized by the fact that: height of said fixation projection (2-5) is smaller than or equal to that of the bottle (2).

4. A removable liquid bottle as described in claim 1, is characterized by the fact that: said projection with a bevel (2-4) of the bottle (2) is located in the upper part of the bottle (2).

5. A removable liquid bottle as described in claim 4, is characterized by the fact that: the bottom of said projection with a bevel (2-4) is an arcuate or flat surface.

6. A removable liquid bottle as described in claim 4, is characterized by the fact that: the bottom of said projection with a bevel (2-4) is a curve with at least two minimum points.

7. A removable liquid bottle as described in claim 1, is characterized by the fact that: said fixation projection (2-5) of the bottle (2) and its opposite front section are not centrosymmetric by the centerline of the bottle (2).

8. A removable liquid bottle as described in claim 1, is characterized by the fact that: said stopper (3) consists of an internal housing (3-1) with an interfixed wick, an external housing (3-2) in the opening of the bottle and an annulus (3-3) to connect the internal housing (3-1) and the external housing (3-2), wherein there is an outlet (3-4) at the top of the internal housing (3-1).

9. A removable liquid bottle as described in claim 1, is characterized by the fact that: said internal housing (3-1) of said stopper (3) is a cone with a narrow top and a wide bottom.
